(19) United States
(12) Patent Application Publication
(10) Pub. No.: US 2010/0152736 A1
(43) Pub. Date: Jun. 17, 2010

(54) OSTEOSYNTHESIS DEVICE FOR HUMERUS NECK FRACTURES AND FRACTURE-DISLOCATIONS

(76) Inventor: Alexander Bukreev, Adigeia (RU)

Correspondence Address: John Alumit, 16830 Ventura Blvd. Suite 360, Encino, CA, 91436, US

(21) Appl. No.: 12/712,092

(22) Filed: Feb. 24, 2010

(30) Foreign Application Priority Data
Aug. 27, 2007 (IB) PCT/IB2008/002224

(51) Int. Cl. A61B 17/56

(52) U.S. Cl. 606/62

(57) ABSTRACT

The invention relates to a device for the osteosynthesis of fractures and luxations of the neck of the humerus. The device is designed as a U-shaped pin consisting of a bioinert metal, said pin being 10 cm long with a distance of 1 cm between the limbs and being curved through a radius of 9 cm along its length. This reduces the traumatism of the soft parts during osteosynthesis, prevents the development of post-operative complications caused by infections and avoids damage to the axillary nerve and dislocation of the fracture fragments after osteosynthesis.

### CROSS-REFERENCE TO RELATED APPLICATION

This application claims the benefit of the priority filing date in PCT PCT/IB2008/002224 referenced in WIPO Publication WO/2009/027797. The earliest priority date claimed is Aug. 27, 2007.

### FEDERALLY SPONSORED RESEARCH

Not Applicable

### SEQUENCE LISTING OR PROGRAM

Not Applicable

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention relates to an osteosynthesis device for humerus neck fractures and fracture-dislocations.

The device relates to medicine, notably in the fields of orthopedics and traumatology. The device can be used for the surgical treatment of different types of humerus neck fractures and fracture-dislocations (Internal osteosynthesis instructions. Technique recommended by AO group (Switzerland) Springer-Verlag, 2001. 650 pages. P. 156-159).

One of the problems of contemporary orthopedics and traumatology is the further development of effective hardware for the surgical treatment of shoulder fractures and fracture-dislocations, in the three fracture types. Fractures of the proximal end of the humerus amount to about 5 percent of all fractures. The highest possible functional result is always planned in surgical treatment. Using well-known existing hardware for post-surgical treatment, there is always a probability for secondary dislocation of fracture fragments and other complications. New hardware developments eliminate some of the disadvantages of the existing hardware. The use of variant model plates does not always eliminate fractures completely. Patients still suffer from pain and shoulder joint dysfunction.

Even when using known hardware, accompanied by an extensive, traumatic, and non-anatomic approach, there is always the possibility of traumatizing muscles, blood vessels and nerves. This results in subsequent secondary illnesses, posttraumatic deforming arthrosis of the brachial joint, and in some cases, osteonecrosis, atrophy of the shoulder girdle, and severe contractures. The application of a completed construction avoids these complications and achieves an earlier and more precise repositioning of the fragments, with secure and nontraumatic internal osteosynthesis. Based on observed data from domestic and foreign literature on surgical treatment of humerus neck fractures and shoulder fracture-dislocations, it can be noted that the most common and effective healing methods are open reposition and internal osteosynthesis. However, among the diversity of existing hardware, there is no single construction that allows a simple, non-bulky, non-traumatic and secure fixation. Known is a device for the osteosynthesis of humerus neck fractures and displaced fracture-dislocations, which uses a T-shaped plate with three or four holes in it (M. E. Mueller. Internal osteosynthesis instructions. Technique recommended by AO group (Switzerland) Springer-Verlag, 2001. 650 pages. P. 156-159).

This plate is used in the following way:

The best access to the proximal part of the humerus is achieved through a lengthwise incision along the sulcus between m. pectoralis and m. deltoideus. The approach can be expanded by the excision of the deltoid muscle from the clavicle and the acromion process.

Rigid fixation is achieved by open reposition and by internal fixation of the plate. The plate has to be placed in such a way as not to disturb the function of the long tendon m. biceps. Spongy bone screws of 6.5 mm, which are threaded along the full length of the plate, are usually fit for head fragment fixation. In subcapital fractures, the diaphysis is always situated subcutaneously and can be placed back under the head with the help of a small bone retractor.

After surgery, the arm is placed in the abduction position to form a splint. Physiotherapy is started in this position. Prolonged immobilization of the shoulder joint must be avoided. The drawbacks of extracortical osteosynthesis of the T-shaped plate are the following:

1) The procedure of plate application demands an extensive surgical approach and bone denudation at large length. These factors increase the danger of the development of infectious complications as compared with intramedullary osteosynthesis or external extrafocal osteosynthesis.
2) The massive plate, placed on periosteum, even without its detachment, leads to periosteal blood supply disturbance. The plate, which comes in contact with the entire bone surface, causes necrosis and generalized osteoporosis. This is the natural, biological reaction of a bone, when its systems are remodeled in an accelerated fashion.
3) If the plate were extracted before the completion of the remodeling processes, refractures in the places of barrier insertions, owing to the disturbance of bone strength qualities connected with osteoporosis, can occur.
4) There is a high probability of axillary nerve (n. axsilaris) injury during the osteosynthesis.

K. M. Klimov's T-shaped plate with a hook-like end represents the nearest prior art (Kaplan A. V. Closed injuries of bones and joints. “Medicine”, Moscow, 1967, pp. 156-157). The fragments of a humerus neck fracture are fixed with the help of a T-shaped plate with a hook-like end. The surgery is performed in the following way. After the fragment approximation, the shoulder is rotated inward. Then, a groove is made with the help of an electric circular saw, 5-6 cm in length. The groove passes through the entire thickness of the cortical layer, downwards from the fracture place, outwards and parallel to the tendon of pectoralis major muscle and long the head of biceps. A beak-shaped end of the T-shaped plate is driven into the head of the biceps by this groove. Then the T-shaped plate is hammered in the sawed out groove of the humerus. The plate is then fixed with two cotters, which are hammered in the plate's holes.

The drawbacks of osteosynthesis with Klimov's T-shaped plate are:

1) Osteosynthesis with the plate demands an extensive surgical approach, which presupposes the excision of the deltoid muscle from the clavicle and extensive preparation of the shoulder's periosteum.
2) This traumatic approach and a plate-involving osteosynthesis, which is traumatic in itself, both create a danger of the development of post-surgical infectious complications.
3) There is a high probability of axillary nerve injury as a result of the extensive approach and traumatic plate-involving osteosynthesis.
(Kaplan A. V. Closed injuries of bones and joints. “Medicine”, Moscow, 1967, pp. 156-157).

Based on the above prior art, the objective of current invention is to create a device for humerus neck fractures and fracture-dislocations, which
decreases the trauma of soft tissues and lessens the extensive preparation of the shoulder's periosteum,
prevents the development of post-surgical infectious complications,
avoids post-surgical injury of the axillary nerve,
eliminates the probability of post-osteosynthesis axial displacement of bone fragments.

The objective is achieved by the features of claim 1.

More expedient and advantageous embodiments of the invention are contained within the subclaims.

The results are achieved through a small sized stud in its original form that has sufficient strength and rigidity. These characteristics allow the stud to pass through the holes during osteosynthesis and into the intramedullary canal. A section of the stud provides strength and rigidity, sufficient for secure fixation at the fracture site. The length of the stud is based on the length of the head of the humerus and the disposition of the holes for stud placement. A stud with a wide cross-section would not go into the intramedullary canal, and a stud with a smaller cross-section would not have sufficient strength and rigidity. The bend angle (concavity) allows for the insertion of the stud into the holes of the bone. Expanions formed on the vertical sides of the stud legs bring an absolute fixation of the proximal and distal ends of the stud in the holes of the bone. The ends of the legs are fixed in the humeral head. The expansions hold both the middle part of the stud on the intramedullary canal walls an the distal end of the stud.

### SUMMARY

The invention relates to a device for the osteosynthesis of fractures and luxations of the neck of the humerus. The device is designed as a U-shaped pin consisting of a bioinert metal, said pin being 10 cm long with a distance of 1 cm between the limbs and being curved through a radius of 9 cm along its length. This reduces the traumatism of the soft parts during osteosynthesis, prevents the development of post-operative complications caused by infections and avoids damage to the axillary nerve and dislocation of the fracture fragments after osteosynthesis.

### BRIEF DESCRIPTION OF THE FIGURES

FIG. 1 shows a frontal plane view of the device and

FIG. 2 shows a horizontal plane view of the device.

### DETAILED DESCRIPTION

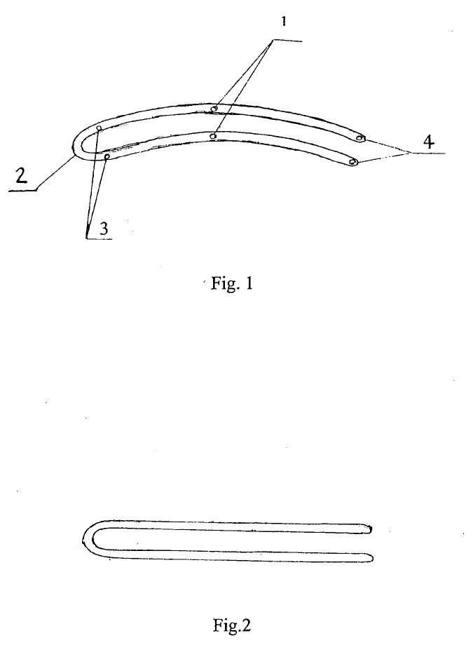

FIG. 1 shows a frontal plane view of the device as claimed with the fixation points in the stud's middle part 1; the proximal stud end, projecting from the bone to about 1 cm, after osteosynthesis 2; fixation points of the stud's proximal part 3; and fixation points of the stud's distal part 4.

FIG. 2 shows a horizontal plane view of the device as claimed.

The device is used as follows: Surgery is performed under intravenous anesthesia, with an anterolateral approach in the upper one-third of the shoulder to the humeral bone in the patient supine position. The arm is adducted to the trunk and the elbow joint is bent at a 90 degree angle, and put on the attached table. Depending on the size of the shoulder, the average dimension of approach is about 12 cm. Approach is made along the fibers of the deltoid muscle (m. deltoidea) via layer-by-layer dissection of the cutis, the hypodermic tissue, the superficial fascia, the shoulder aponeurosis (fascia brachii) and the front the deltoid muscle. The deltoid muscle is moved apart along its fibers for 3 cm with the help of a soft dissecting clamp. Then, using two fingers of both hands, the muscle is detached along the full length of approach to the subdeltoid space (bursa subdeltoidea). The fracture site is then fronted. The place for the stud's insertion is made ready. The bone is prepared from the periosteum, diverging from the fracture line of the surgical shoulder neck on the lateral surface of the humeral bone to the attaching point of the deltoid muscle. The “triangle” is formed on the bone with the fracture site as the basis; its right side being on the medial edge (margo medialis) and its left side being on the lateral edge (margo lateralis). Then, with the help of a single-tooth retractor, traction is performed along the shoulder axis with the elbow joint bent at a 90 degree angle. Fracture reduction is reached by rotary movements. In the upper one-third of the lateral shoulder zone (corpus humeri) close to the vertex of the “triangle,” two holes are formed transversally at a 1 cm distance toward the humeral head (caput humeri) at a sharp angle toward the bone to the intramedullary canal. The U-shaped stud is inserted in the holes; the stud has a rectangular cross-section of 0.2-0.3 cm and congruentially adjoins the humeral bone. The bend angle of the stud allows for the insertion of the stud through the holes in the bone. Due to the bend angle, the stud glides along the back side of the intramedullary canal and enters the head fixed with both legs. The stud is inserted in the humeral head under the R-control. After osteosynthesis, the stud's proximal end projects from the bone for 1 cm. Sutures are placed on the injury layer-by-layer. An aseptic dressing is applied. Part of the shoulder that underwent surgery is immobilized in plaster Desault's bandage for a month. After bandage removal, contracture work-out is prescribed with the help of therapeutic physical training until functional recovery between 2-3 weeks.

The result of therapy is satisfactory. Limb function is almost completely recovered.

All features disclosed in this specification, including any accompanying claims, abstract, and drawings, may be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means for” performing a specified function, or “step for” performing a specific function, is not to be interpreted as a “means” or “step” clause as specified in 35 U.S.C. §112, paragraph 6. In particular, the use of “step of” in the claims herein is not intended to invoke the provisions of 35 U.S.C. §112, paragraph 6.

Although preferred embodiments of the present invention have been shown and described, various modifications and substitutions may be made thereto without departing from the spirit and scope of the invention. Accordingly, it is to be understood that the present invention has been described by way of illustration and not limitation.

What is claimed is:

1. An osteosynthesis device for humerus neck fractures and fracture-dislocations characterized in that it is a U-shaped stud made of bioinert metal, 10 cm in length with a distance between the legs of 1 cm, wherein the stud is concaved through the whole length with a radius of 9 cm.

2. The device as defined by claim 1, characterized in that the stud legs feature a rectangular cross-section of about 0.2-0.3 cm.

3. The device as defined by claim 1, characterized in that the stud legs are curved at the proximal end (2), wherein the radius of curvature corresponds to about half the distance between the stud legs.

4. The device as defined by claim 2, characterized in that the stud legs are curved at the proximal end (2), wherein the radius of curvature corresponds to about half the distance between the stud legs.

5. The device as defined by claims 1, characterized in that expansions are formed on the vertical sides of the stud legs between the proximal end (2) in the middle part and the distal part of stud fixation elements (3, 1 and 4).

6. The device as defined by claims 2, characterized in that expansions are formed on the vertical sides of the stud legs between the proximal end (2) in the middle part and the distal part of stud fixation elements (3, 1 and 4).

7. The device as defined by claims 3, characterized in that expansions are formed on the vertical sides of the stud legs between the proximal end (2) in the middle part and the distal part of stud fixation elements (3, 1 and 4).

8. The device as defined by claims 4, characterized in that expansions are formed on the vertical sides of the stud legs between the proximal end (2) in the middle part and the distal part of stud fixation elements (3, 1 and 4).

9. The device as defined by claim 5, characterized in that the formed expansions (3, 1 and 4) for stud fixation feature a circular cross-section, wherein the radius of the circular cross-sections (3, 1 and 4) is equivalent to about half the thickness of the legs of the stud.

10. The device as defined by claim 6, characterized in that the formed expansions (3, 1 and 4) for stud fixation feature a circular cross-section, wherein the radius of the circular cross-sections (3, 1 and 4) is equivalent to about half the thickness of the legs of the stud.

11. The device as defined by claim 7, characterized in that the formed expansions (3, 1 and 4) for stud fixation feature a circular cross-section, wherein the radius of the circular cross-sections (3, 1 and 4) is equivalent to about half the thickness of the legs of the stud.

12. The device as defined by claim 8, characterized in that the formed expansions (3, 1 and 4) for stud fixation feature a circular cross-section, wherein the radius of the circular cross-sections (3, 1 and 4) is equivalent to about half the thickness of the legs of the stud.
